(19) United States
(12) Patent Application Publication
(10) Pub. No.: US 2023/0121157 A1
(43) Pub. Date: Apr. 20, 2023

(54) ANTI-AGING CARE COMPOSITION COMPRISING PERFUMES

(71) Applicant: BASF SE, Ludwigshafen am Rhein (DE)

(72) Inventors: Myriam SOHN, Grenzach-Wyhlen (DE); Marcel SCHNYDER, Grenzach-Wyhlen (DE); Stanislaw KRUS, Grenzach-Wyhlen (DE); Stephanie ACKER, Grenzach-Wyhlen (DE)

(21) Appl. No.: 17/910,371

(22) PCT Filed: Mar. 11, 2021
(86) PCT No.: PCT/EP2021/056163

§ 371 Date: Sep. 9, 2022

(30) Foreign Application Priority Data
Mar. 13, 2020 (EP) 20163051.4

(51) Int. Cl. A61K 8/44; A61Q 19/08; A61Q 13/00; A61Q 17/04

(52) U.S. Cl. CPC A61K 8/44; A61Q 19/08; A61Q 13/00; A61Q 17/04

(57) ABSTRACT

The present invention relates to the use of diethylamino hydroxybenzoyl hexyl benzoate in an anti-aging care composition comprising at least one perfume and to a body-care composition comprising diethylamino hydroxybenzoyl hexyl benzoate and at least one perfume for use in a method of preventing skin reaction.

The present invention relates to the use of diethylamino hydroxybenzoyl hexyl benzoate in an anti-aging care composition comprising at least one perfume.

UV radiation causes harmful effects on the human skin. Beside the acute effect of sunburn of the skin, UV radiation is also known to increase the risk of skin cancer. Furthermore, long time exposure to UV-A and UV-B light can cause phototoxic and photo allergenic reactions on the skin and can accelerate skin aging. Hence, UV filters are suitable ingredients for anti-aging care composition.

To protect the human skin from UV radiation, various sun protecting UV filters (also referred to as UV absorbers) exist including UV-A filter, UV-B filter, and broadband filters. These filters are added to e.g. sunscreen or cosmetic compositions. The UV filters are either organic or inorganic, particulate or non-particulate compounds, of which all have a high absorption efficacy in the UV-light range. In general, UV light can be divided into UV-A radiation (320-400 nm) and UV-B radiation (280-320 nm). Depending on the position of the absorption maxima, UV filters are divided into UV-A and UV-B filters. In case an UV filter absorbs both, UV-A and UV-B light, it is referred to as a broadband absorber.

Since 2006, the EU commission has recommended that all sunscreen or cosmetic compositions should have an UV-A protection factor, which is at least one third of the labelled sun protection factor (SPF), wherein the sun protection factor refers mainly to the UV-B protection.

2-(4′-Diethylamino-2′-hydroxybenzoyl)benzoic acid hexyl ester (also referred to as diethylamino hydroxybenzoyl hexyl benzoate or DHHB) is an effective UV-A filter and is used to protect skin from UV radiation (e.g. in sunscreen). 2-(4′-Diethylamino-2′-hydroxybenzoyl)benzoic acid hexyl ester is depicted below, and will in the following also be referred to as compound of formula (I).

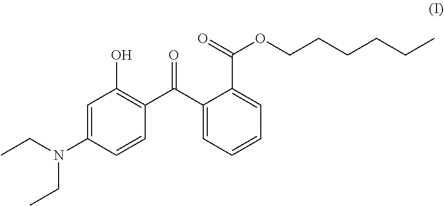 [SMILES: CCCCCCOC(=O)c1ccccc1C(=O)c1ccc(N(CC)CC)cc1O]

EP2276452 and EP2276449 relate to cosmetic preparations comprising DHHB and at least one perfume in order to examine the odor stability.

Anti-aging care compositions often additionally comprise perfumes contributing to a pleasant odor. Perfumes however tend to react to free radicals when exposed to UV radiation. This may trigger a radical chain reaction leading to destabilization of the formulation itself. Thus, the efficacy of an UV filter in such destabilized formulation is also reduced in view of protection against UV irradiation. Furthermore, the presence of hydroperoxide and other peroxide free radicals are known to induce contact dermatosis and severe allergic reactions (Bjorkman et al., Contact Dermatitis, 70, 2014, 129-138).

Since anti-aging care compositions are often applied to body parts which are exposed directly to UV radiation, there is an ongoing need to provide an anti-aging care composition comprising at least one perfume, which can safely be applied on body parts, which are exposed to UV radiation. In this connection, it has been an object of the present invention to provide an anti-aging care composition comprising at least one perfume having a reduced formation of free radicals. Further, it has been an object of the present invention to provide an anti-aging care composition comprising at least one perfume having a reduced lipid peroxidation. Finally, it has been an object of the present invention to provide a body-care composition for use in a method of preventing skin reaction caused by perfumes.

It has surprisingly been found that at least one of the above objects can be achieved by the use of diethylamino hydroxybenzoyl hexyl benzoate in a body-care composition comprising at least one perfume.

In particular, the inventors of the present invention have found that when DHHB is used in an anti-aging care composition comprising at least one perfume the oxidative degradation is reduced.

The present invention therefore relates in a first aspect to the use of diethylamino hydroxybenzoyl hexyl benzoate in an anti-aging care composition comprising at least one perfume.

In the following, preferred embodiments of the use are described in further detail. It is to be understood that each preferred embodiment is relevant on its own as well as in combination with other preferred embodiments.

In a preferred embodiment A1 of the first aspect, the diethylamino hydroxybenzoyl hexyl benzoate in said anti-aging care composition comprising at least one perfume is used for reducing the oxidative degradation.

In a preferred embodiment A2 of the first aspect, the diethylamino hydroxybenzoyl hexyl benzoate in said anti-aging care composition comprising at least one perfume is used for reducing the lipid peroxidation.

In a preferred embodiment A3 of the first aspect, the anti-aging care composition further comprises butyl methoxydibenzoylmethane.

In a preferred embodiment A4 of the first aspect, the anti-aging care composition further comprises at least one additional UV filter.

In a preferred embodiment A5 of the first aspect, the at least one perfume is selected from the group consisting of limonene, citral, linalool, alpha isomethyl ionone, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzyl alcohol, benzyl benzoate, benzylcinnamate, benzyl salicylate, bergamot oil, orange oil, bitter orange oil, butylphenylmethylpropioal, cardamom oil, cedrol, cinnamaldehyde, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexyl cinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin, and mixtures thereof.

In a preferred embodiment A6 of the first aspect, the at least one perfume is selected from the group consisting of the at least one perfume is selected from the group consisting of geraniol, linalool, hydroxycitronellal, alpha isomethyl ionone, citral, coumarin, limonene, citronellol, benzyl benzoate, benzyl salicylate, hexyl cinnamaldehyde, vanillin, eugenol, orange oil, benzyl alcohol, bergamot oil, cinnamaldehyde, and mixtures thereof, and in particular wherein the at least one perfume is selected from the group consisting of geraniol, alpha isomethyl ionone, citral, coumarin, benzyl salicylate, hexyl cinnamaldehyde, eugenol, and mixtures thereof.

In a preferred embodiment A7 of the first aspect, the at least one perfume is selected from the group consisting of geraniol, linalool, alpha isomethyl ionone, citral, coumarin, citronellol, benzyl salicylate, hexyl cinnamaldehyde, vanillin, orange oil, benzyl alcohol, bergamot oil, cinnamaldehyde, and mixtures thereof, preferably wherein the at least one perfume is selected from the group consisting of alpha isomethyl ionone, citral, coumarin, citronellol, benzyl salicylate, hexyl cinnamaldehyde, vanillin, orange oil, cinnamaldehyde, and mixtures thereof, more preferably wherein the least one perfume is selected from the group consisting of alpha isomethyl ionone, citral, coumarin, benzyl salicylate, hexyl cinnamaldehyde, vanillin, cinnamaldehyde, bitter orange oil, and mixtures thereof, and in particular wherein the at least one perfume is selected from the group consisting of alpha isomethyl ionone, citral, hexyl cinnamaldehyde, and mixtures thereof.

In a preferred embodiment A8 of the first aspect, the anti-aging care composition comprises hexyl cinnamaldehyde, diethylamino hydroxybenzoyl hexyl benzoate, optionally butyl methoxydibenzoylmethane, and optionally at least one additional UV filter.

In a preferred embodiment A9 of the first aspect, the anti-aging care composition comprises the at least one perfume, diethylamino hydroxybenzoyl hexyl benzoate, butyl methoxydibenzoylmethane, and optionally at least one additional UV filter.

In a preferred embodiment A10 of the first aspect, the anti-aging care composition comprises the at least one perfume in an amount of from 0.00001 to 1 wt.-%, preferably from 0.001 to 0.5 wt.-%, based on the total weight of the anti-aging care composition; diethylamino hydroxybenzoyl hexyl benzoate in an amount of from 0.1 to 10 wt.-%, preferably from 0.1 to 8 wt.-%, based on the total weight of the anti-aging care composition; optionally butyl methoxydibenzoylmethane in an amount of from 0.1 to 5 wt.-%, preferably from 0.1 to 4 wt.-%, based on the total weight of the anti-aging care composition; and optionally at least one additional UV filter in an amount of from 0.1 to 10 wt.-%, preferably from 0.1 to 8 wt.-%, based on the total weight of the anti-aging care composition.

In a preferred embodiment A11 of the first aspect, the anti-aging care composition comprises the at least one perfume in an amount of from 0.001 to 0.5 wt.-%, based on the total weight of the anti-aging care composition;

diethylamino hydroxybenzoyl hexyl benzoate in an amount of from 0.1 to 8 wt .-%, based on the total weight of the anti-aging care composition;

butyl methoxydibenzoylmethane in an amount of from 0.1 to 4 wt.-%, based on the total weight of the anti-aging care composition;

optionally at least one additional UV filter in an amount of from 0.1 to 8 wt .-%, based on the total weight of the anti-aging care composition.

In a preferred embodiment A12 of the first aspect, the anti-aging care composition further comprises a dermatologically acceptable emulsifier, thickener, and/or emollient.

In a preferred embodiment A13 of the first aspect, the anti-aging care composition is free of polyethylene glycol and polypropylene glycol.

In a preferred embodiment A14 of the first aspect, the anti-aging care composition is free of ethylhexyl methoxycinnamate and octocrylene.

In a preferred embodiment A15 of the first aspect, the diethylamino hydroxybenzoyl hexyl benzoate in said anti-aging care composition comprising at least one perfume is used for reducing the lipid peroxidation, which is triggered by UV radiation.

In a preferred embodiment A16 of the first aspect, the diethylamino hydroxybenzoyl hexyl benzoate in said anti-aging care composition comprising at least one perfume is used for reducing the aldehyde formation, which is triggered by UV radiation, in particular for reducing the formation of malondialdehyde.

The present invention relates in a second aspect to a body-care composition comprising diethylamino hydroxybenzoyl hexyl benzoate and at least one perfume for use in a method of preventing dermatosis and/or allergic reactions caused by perfumes.

### DETAILED DESCRIPTION

Before describing in detail exemplary embodiments of the present invention, definitions important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The term “compound of formula (I)” refers to 2-(4′-diethylamino-2′-hydroxybenzoyl)benzoic acid hexyl ester (also known as diethylamino hydroxybenzoyl hexyl benzoate or DHHB).

The term “body-care composition” refers to any product suitable to apply to the human body, e.g. anti-aging care composition, sunscreen compositions, bath and shower products, preparations containing fragrances and odoriferous substances, hair-care products, dentifrices, decorative preparations, and cosmetic formulations containing active ingredients. Preferred are sunscreen compositions.

The term “sunscreen composition” or “sunscreen” refers to any topical product, which reflects and/or absorbs certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic UV filters, as well as other ingredients or additives, e.g., emulsifiers, emollients, viscosity regulators, stabilizers, preservatives, or perfumes (fragrances).

The term “anti-aging care composition” refers to any topical product, which absorbs and which may further reflect and scatter certain parts of UV radiation and is used as an everyday care product for the human body, e.g., for face, body or hair. The anti-aging care composition may comprise one or more active agents, e.g., organic or inorganic UV filters, as well as other ingredients or additives, e.g., emulsifiers, emollients, viscosity regulators, stabilizers, preservatives, or perfumes (fragrances). It is to be understood that the anti-aging care composition according to the present invention provides for reduction of oxidative degeneration and limits the skin aging process induced by free radicals.

Suitable bath and shower additives are, e.g., shower gels, bath-salts, bubble baths and soaps.

Suitable preparations containing fragrances and odoriferous substances are in particular scents, perfumes, toilet waters and shaving lotions (aftershave preparations).

Suitable hair-care products are, e.g., shampoos for humans and animals, in particular dogs, hair conditioners, products for styling and treating hair, perming agents, hair sprays and lacquers, hair gels, hair fixatives and hair dyeing or bleaching agents.

Suitable dentifrices are, e.g., tooth creams, toothpastes, mouth-washes, mouth rinses, anti-plaque preparations and cleaning agents for dentures.

Suitable decorative preparations are, e.g., lipsticks, nail varnishes, eye shadows, mascaras, dry and moist make-up, rouge, powders, depilatory agents and suntan lotions.

Suitable cosmetic formulations containing active ingredients are, e.g., hormone preparations, vitamin preparations, vegetable extract preparations and antibacterial preparations.

The cited body-care compositions can be in the form of creams, ointments, pastes, foams, gels, lotions, powders, make-ups, sprays, sticks or aerosols. They preferably contain the compound of formula (I) in the lipophilic phase.

The term “emollient” relates to cosmetic preparations used for protecting, moisturizing and lubricating the skin. The word emollient is derived from the Latin word mollire, to soften. In general, emollients prevent evaporation of water from the skin by forming an occlusive coating. They can be divided into different groups depending on their polarity index.

The term “photostability” refers to the ability of a UV filter or any other molecule, which is exposed to sunlight, to stay stable upon irradiation. In particular, this means that the compound does not undergo a degradation process upon UV radiation.

The term “critical wavelength” is defined as the wavelength at which the area under the UV protection curve (% protection versus wavelength) represents 90% of the total area under the curve in the UV region (280-400 nm). For example, a critical wavelength of 370 nm indicates that the protection of the sunscreen composition is not limited to the wavelengths of UV-B, i.e. wavelengths from 280-320 nm, but extends to 370 nm in such a way that 90% of the total area under the protective curve in the UV region are reached at 370 nm.

The term “ultraviolet filter” or “UV filter” as used herein refers to organic or inorganic compounds, which can absorb and/or reflect UV radiation caused by sunlight. UV filter can be classified based on their UV protection curve as UV-A, UV-B or broadband filters. In the context of the present application, broadband filters may be listed as UV-A filters, as they also provide UV-A protection. In other words, preferred UV-A filters also include broadband filters.

The definition of “broadband” protection (also referred to as broad-spectrum or broad protection) is based on the “critical wavelength”. For broadband coverage, UV-B and UV-A protection must be provided. According to the US requirements, a critical wavelength of at least 370 nm is required for achieving broad spectrum protection. Furthermore, it is recommended by the European Commission that all sunscreen or cosmetic compositions should have an UVA protection factor, which is at least one third of the labelled sun protection factor (SPF), e.g. if the sunscreen composition has an SPF of 30 the UVA protection factor has to be at least 10.

Preferred embodiments regarding the use according to the present invention are described hereinafter. It is to be understood that the preferred embodiments of the invention are preferred alone or in combination with each other.

As indicated above, the present invention relates in one embodiment to the use of diethylamino hydroxybenzoyl hexyl benzoate in an anti-aging care composition comprising at least one perfume.

Preferred embodiments regarding the use are defined hereinafter.

The diethylamino hydroxybenzoyl hexyl benzoate, which is according to the present invention used in the anti-aging care composition comprising at least one perfume may be comprised in the anti-aging care composition in an amount of from 0.1 to 10 wt.-%, preferably from 0.1 to 9 wt.-%, more preferably from 0.1 to 8 wt.-%, even more preferably from 0.1 to 7 wt.-% or from 0.1 to 6 wt.-% or from 0.1 to 5 wt.-%, in particular from 0.1 to 4 wt.-%, based on the total weight of the anti-aging care composition.

In one embodiment of the present invention, the diethylamino hydroxybenzoyl hexyl benzoate in said anti-aging care composition comprising at least one perfume is used for reducing oxidative degradation. Oxidation of sensitive ingredients (i.e. oxidative degradation) following an UV irradiation may happen in the anti-aging care composition or the applied film comprising at least one perfume. In this connection it is to be understood that the generated free radicals may further react with compounds present e.g. in the formulation or on the skin such as ingredients from the sebum or with sensitive skin molecules and produce undesirable skin reactions caused by perfumes.

In one embodiment of the present invention, the diethylamino hydroxybenzoyl hexyl benzoate in said anti-aging care composition comprising at least one perfume is used for reducing oxidative degradation in the formulation or on the skin.

In one embodiment of the present invention, the diethylamino hydroxybenzoyl hexyl benzoate in said anti-aging care composition comprising at least one perfume is used for reducing lipid peroxidation. In this connection it is to be understood that the formation of lipid peroxides or lipid oxidation products (LOPs) is reduced. Further, according to one embodiment of the present invention, the diethylamino hydroxybenzoyl hexyl benzoate in said anti-aging care composition comprising at least one perfume is used for reducing damage to the cell membrane.

In one embodiment of the present invention, the anti-aging care composition further comprises butyl methoxydibenzoylmethane. Butyl methoxydibenzoylmethane (also known as 4-(tert.-butyl)-4′-methoxydibenzoylmethane or BMDBM) may be comprised in the anti-aging care composition in an amount of from 0.1 to 5 wt.-%, preferably from 0.2 to 4.5 wt.-%, more preferably from 0.3 to 3 wt.-%, and in particular from 0.5 to 2 wt.-%, based on the total weight of the anti-aging care composition.

In one embodiment of the present invention, the anti-aging care composition further comprises at least one additional UV filter. The at least one additional UV filer may be comprised in the anti-aging care composition in an amount of from 0.1 to 10 wt.-%, preferably from 0.2 to 9 wt.-%, more preferably from 0.3 to 8 wt.-%, even more preferably from 0.4 to 7 wt.-% or from 0.4 to 6 wt.-%, and in particular from 0.5 to 5 wt.-%, based on the total weight of the anti-aging care composition.

The at least one additional UV filter may be selected from the group consisting of UV-A filter, UV-B filter, broadband filter, and mixtures thereof. Suitable UV filter may be organic or inorganic, particulate or non-particulate compounds.

In one embodiment of the present invention, the at least one additional UV filter is an organic UV filter.

In another embodiment of the present invention, the at least one additional UV filter is an inorganic UV filter. Suitable inorganic UV filter may exemplarily be zinc oxide and titanium dioxide.

In another embodiment of the present invention, the anti-aging care composition comprises at least one organic UV filter and at least one inorganic UV filter.

In one embodiment of the present invention, the at least one additional UV filter is selected from the group consisting of triazine derivatives, dibenzoylmethane derivatives, benzotriazole derivatives, benzimidazole derivatives and combinations thereof.

In a preferred embodiment, the at least one further UV filter is a UV-A filter, preferably selected from the group consisting of 2,4-bis-{[4-(2-ethyl-hexyloxy)-2-hydroxy]-phenyl}-6-(4-methoxyphenyl)-1,3,5 triazine (INCI bis-ethylhexyloxyphenol methoxyphenyl triazine), 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane), 2-(2H-benzotriazol-2-yl)-6-(2-ethyl hexyloxymethyl)-4-methyl-phenol, methylene bis-benzotriazolyl tetramethylbutylphenol, terephthalylidene dicamphor sulfonic acid, and combinations thereof.

In another preferred embodiment, the at least one further UV filter is a UV-B filter, preferably selected from the group consisting of 4,4′,4″-(1,3,5-triazin-2,4,6-triyltriimino)tris-benzoic acid-tris(2-ethylhexyl)ester (INCI ethylhexyl triazone), malonate derivatives such as dimethicone diethyl benzalmalonate, diethylhexyl butamido triazone, phenylbenzimidazole sulfonic acid, and combinations thereof.

In one embodiment of the present invention, the at least one additional UV filter is selected from the group consisting of 2,4-bis-{[4-(2-ethyl-hexyloxy)-2-hydroxy]-phenyl}-6-(4-methoxyphenyl)-1,3,5 triazine (INCI bis-ethylhexyloxyphenol methoxyphenyl triazine), 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane), 2-(2H-benzotriazol-2-yl)-6-(2-ethyl hexyloxymethyl)-4-methyl-phenol, 4,4′,4″-(1,3,5-triazine-2,4,6-triyltriimino)tris-benzoic acid-tris(2-ethylhexyl)ester (INCI ethylhexyl triazone), malonate derivatives such as dimethicone diethyl benzalmalonate, diethylhexyl butamido triazone, 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester, [(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI terephthalylidene dicamphor sulfonic acid), disodium phenyl dibenzimidazole tetrasulfonate, phenylbenzimidazole sulfonic acid, and the combination thereof.

In one embodiment of the present invention, the at least one additional UV filter is selected from the group consisting of 2,4-bis-{[4-(2-ethyl-hexyloxy)-2-hydroxy]-phenyl}-6-(4-methoxyphenyl)-1,3,5 triazine (INCI bis-ethylhexyloxyphenol methoxyphenyl triazine), 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone), and mixtures thereof.

In one embodiment of the present invention, the at least one additional UV filter is selected from the group consisting of [(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI terephthalylidene dicamphor sulfonic acid), disodium phenyl dibenzimidazole tetrasulfonate, phenylbenzimidazole sulfonic acid, and the combination thereof.

Any suitable perfume may be used as the at least one perfume according to the present invention. The at least one perfume may be comprised in the anti-aging care composition in an amount of from 0.00001 to 1 wt.-%, preferably from 0.0001 to 0.9 wt.-%, more preferably from 0.001 to 0.8 wt.-% or from 0.001 to 0.7 wt.-%, and in particular from 0.01 to 0.6 wt.-%, based on the total weight of the anti-aging care composition.

In one embodiment of the present invention, the at least one perfume is selected from the group consisting of limonene (also known as 1-methyl-4-(prop-1-en-2-yl)cyclohex-1-ene), citral (also known as 3,7-dimethylocta-2,6-dienal), linalool (also known as 3,7-dimethyl-1,6-octadien-3-01), alpha isomethyl ionone (also known as alpha cetone), geraniol (also known as (2E)-3,7-dimethyl-2,6-octadien-1-ol), citronellol (also known as 3,7-dimethyloct-6-en-1-ol), 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzyl alcohol, benzyl benzoate, benzylcinnamate, benzyl salicylate (also known as benzyl 2-hydroxybenzoate), bergamot oil, orange oil, bitter orange oil, butylphenylmethylpropioal, cardamom oil, cedrol, cinnamaldehyde (also known as (2E)-3-Phenylprop-2-enal), cinnamylalcohol, citronnellylmethylcrotonate, lemon oil, coumarin (also known as 2H-chromen-2-one), diethylsuccinate, ethyllinalool, eugenol (also known as 2-methoxy-4-(prop-2-en-1-yl)phenol), evernia furfuracea extracte, evernia prunastri extracte, farensol, guajak wood oil, hexyl cinnamal (also known as hexyl cinnamaldehyde or 2-benzylideneoctanal), hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin (also known as 4-hydroxy-3-methoxybenzaldehyde), and mixtures thereof.

In one embodiment of the present invention, the at least one perfume is a sensitive perfume. In this connection it is to be understood that the term “sensitive perfume” denotes perfumes which are sensitive to oxidative degradation, in particular which are sensitive to oxidative degradation, which is triggered by UV radiation. The sensitivity may be determined using the following test:

To measure the free radicals generation inside a formulation after UV irradiation, the concentration of a photostable spin marker added to the tested samples is followed by Electron Spin Resonance (ESR) spectroscopy before and after UV irradiation of the tested samples. The photostable spin marker will promptly react with the free radical generated in the formulation after UV irradiation.

0.1% (w/w) of the perfume to be tested is incorporated into the formulation, the formulation is then diluted in distillated water (1:10 dilution factor) and a water solution of the spin marker PCA (2,2,5,5,-tetramethyl pyrrolidine N-oxyl) is added to obtain a spin marker concentration of 0.01 mM. Knowing the concentration of the spin trap PCA within the formulation, the amount of reduced PCA can be calculated (Herrling et al., SOFW-Journal, 5-2014, 140, 10-16). The sample is inserted in a capillary quartz tube to perform ESR measurement before and after 20 minutes of UV irradiation. The irradiances as integrated value over the spectral ranges of the solar simulator can exemplarily be E=23,5W/m² for UVB and 180W/m² for UVA.

The percentage of UV-induced free radicals inside the formulation containing the perfume to be tested serves as indicator to the sensitivity of said perfume. According to the above described test, a formulation comprising a sensitive perfume contains at least 1% preferably at least 2%, more preferably at least 3%, even more preferably at least 4%, even more preferable at least 5%, even more preferably at least 10%, even more preferably at least 15%, even more preferably at least 20%, and in particular at least 50% of UV-induced free radicals after 20 minutes of UV irradiation.

In one embodiment of the present invention, the at least one perfume is selected from the group consisting of geraniol, linalool, hydroxycitronellal, alpha isomethyl ionone, citral, coumarin, limonene, citronellol, benzyl benzoate, benzyl salicylate, hexyl cinnamaldehyde, vanillin, eugenol, orange oil, benzyl alcohol, bergamot oil, cinnamaldehyde, and mixtures thereof.

In one embodiment of the present invention, the at least one perfume is selected from the group consisting of geraniol, linalool, alpha isomethyl ionone, citral, coumarin, citronellol, benzyl salicylate, hexyl cinnamaldehyde, vanillin, orange oil, benzyl alcohol, bergamot oil, cinnamaldehyde, and mixtures thereof. In one embodiment of the present invention, the at least one perfume is selected from the group consisting of alpha isomethyl ionone, citral, coumarin, citronellol, benzyl salicylate, hexyl cinnamaldehyde, vanillin, orange oil, cinnamaldehyde, and mixtures thereof.

In a preferred embodiment of the present invention, the at least one perfume is selected from the group consisting of alpha isomethyl ionone, citral, coumarin, benzyl salicylate, hexyl cinnamaldehyde, vanillin, cinnamaldehyde, bitter orange oil, and mixtures thereof.

In one embodiment of the present invention, the at least one perfume is selected from the group consisting of alpha isomethyl ionone, citral, coumarin, benzyl salicylate, hexyl cinnamaldehyde, vanillin, cinnamaldehyde, and mixtures thereof. In one embodiment of the present invention, the at least one perfume is selected from the group consisting of alpha isomethyl ionone, citral, benzyl salicylate, hexyl cinnamaldehyde, vanillin, and mixtures thereof.

In one embodiment of the present invention, the at least one perfume is selected from the group consisting of alpha isomethyl ionone, hexyl cinnamaldehyde, vanillin, and mixtures thereof. In one embodiment of the present invention, the at least one perfume is selected from the group consisting of hexyl cinnamaldehyde, vanillin, and mixtures thereof.

In a preferred embodiment of the present invention, the at least one perfume is selected from the group consisting of geraniol, alpha isomethyl ionone, citral, coumarin, benzyl salicylate, hexyl cinnamaldehyde, eugenol, and mixtures thereof.

In one embodiment of the present invention, the at least one perfume is selected from the group consisting of alpha isomethyl ionone, citral, hexyl cinnamaldehyde, and mixtures thereof. In one embodiment of the present invention, the at least one perfume is hexyl cinnamaldehyde. In another embodiment of the present invention the at least one perfume is alpha isomethyl ionone. In another embodiment of the present invention, the at least one perfume is citral. In another preferred embodiment of the present invention, the at least one perfume is geraniol. In another preferred embodiment of the present invention, the at least one perfume is coumarin. In another preferred embodiment of the present invention, the at least one perfume is benzyl salicylate. In another preferred embodiment of the present invention, the at least one perfume is eugenol.

In one embodiment of the present invention the at least one perfume is not selected from the group consisting of linalool, hexyl cinnamal, benzyl salicylate, and citronellol.

In one embodiment of the present invention, the anti-aging care composition comprises hexyl cinnamaldehyde, diethylamino hydroxybenzoyl hexyl benzoate, optionally butyl methoxydibenzoylmethane, and optionally at least one additional UV filter.

In another embodiment of the present invention, the anti-aging care composition comprises alpha isomethyl ionone, diethylamino hydroxybenzoyl hexyl benzoate, optionally butyl methoxydibenzoylmethane, and optionally at least one additional UV filter.

In another embodiment of the present invention, the anti-aging care composition comprises citral, diethylamino hydroxybenzoyl hexyl benzoate, optionally butyl methoxydibenzoylmethane, and optionally at least one additional UV filter.

In one embodiment of the present invention, the anti-aging care composition comprises hexyl cinnamaldehyde, diethylamino hydroxybenzoyl hexyl benzoate, butyl methoxydibenzoylmethane, and optionally at least one additional UV filter.

In one embodiment of the present invention, the anti-aging care composition comprises the at least one perfume, diethylamino hydroxybenzoyl hexyl benzoate, butyl methoxydibenzoylmethane, and optionally at least one additional UV filter.

In one embodiment of the present invention, the anti-aging care composition comprises the at least one perfume in an amount of from 0.00001 to 1 wt.-%, preferably from 0.001 to 0.5 wt.-%, based on the total weight of the anti-aging care composition; diethylamino hydroxybenzoyl hexyl benzoate in an amount of from 0.1 to 10 wt.-%, preferably from 0.1 to 8 wt.-%, based on the total weight of the anti-aging care composition; optionally butyl methoxydibenzoylmethane in an amount of from 0.1 to 5 wt.-%, preferably from 0.1 to 4 wt.-%, based on the total weight of the anti-aging care composition; and optionally at least one additional UV filter in an amount of from 0.1 to 10 wt.-%, preferably from 0.1 to 8 wt.-%, based on the total weight of the anti-aging care composition.

In one embodiment of the present invention, the anti-aging care composition comprises the at least one perfume in an amount of from 0.0001 to 1 wt.-%, preferably from 0.001 to 0.5 wt.-%, based on the total weight of the anti-aging care composition; diethylamino hydroxybenzoyl hexyl benzoate in an amount of from 0.1 to 8 wt.-%, preferably from 0.1 to 7 wt.-%, based on the total weight of the anti-aging care composition; optionally butyl methoxydibenzoylmethane in an amount of from 0.1 to 4 wt.-%, preferably from 0.1 to 3 wt.-%, based on the total weight of the anti-aging care composition; and optionally at least one additional UV filter in an amount of from 0.1 to 8 wt.-%, preferably from 0.1 to 6 wt.-%, based on the total weight of the anti-aging care composition.

In one embodiment of the present invention, the anti-aging care composition comprises the at least one perfume in an amount of from 0.001 to 0.5 wt.-%, based on the total weight of the anti-aging care composition;

diethylamino hydroxybenzoyl hexyl benzoate in an amount of from 0.1 to 8 wt.-%, based on the total weight of the anti-aging care composition;

butyl methoxydibenzoylmethane in an amount of from 0.1 to 4 wt.-%, based on the total weight of the anti-aging care composition;

optionally at least one additional UV filter in an amount of from 0.1 to 8 wt.-%, based on the total weight of the anti-aging care composition.

In one embodiment of the present invention, the anti-aging care composition comprises the at least one perfume in an amount of from 0.01 to 0.4 wt.-%, based on the total weight of the anti-aging care composition;

diethylamino hydroxybenzoyl hexyl benzoate in an amount of from 0.1 to 5 wt.-%, based on the total weight of the anti-aging care composition;

butyl methoxydibenzoylmethane in an amount of from 0.1 to 3 wt.-%, based on the total weight of the anti-aging care composition;

optionally at least one additional UV filter in an amount of from 0.1 to 8 wt.-%, based on the total weight of the anti-aging care composition.

In one embodiment of the present invention, the anti-aging care composition further comprises at least one additional anti-aging ingredient. In this connection, alpha-lipoic acid, ceramides, collagen, coenzyme Q10, disodium uridine phosphate, ethyl ferulate, folic acid, glycolic acid, helianthus annuus seed oil, hyaluronic acid, jojoba oil, lactic acid, niacinamide, polyglyceryl-5 trioleate, retinol, rosmarinus officinalis, rosmarinus officinalis extract, and Vitamin C may be named.

In one embodiment of the present invention, the anti-aging care composition further comprises a dermatologically acceptable emulsifier, thickener, and/or emollient.

Preferred emulsifiers include
glucose derivatives such as cetearyl glucoside, arachidyl glucoside, lauryl glucoside, polyglyceryl-3 methylglucose distearate, methyl glucose sesquistearate;
sucrose derivative such as sucrose polystearate, sucrose palmitate;
sorbitol derivatives;
glycerides of fatty acids such as glyceryl stearate, glyceryl oleate;
glumatic acid derivatives such as sodium stearoyl glutamate;
sulfosuccinic acid derivatives such as disodium cetearyl sulfosuccinate;
phosphoric acid derivatives such as potassium cetyl phosphate;
fatty acid esters of polyglyceryl such as polyglyceryl-3-diisostearate, polyglyceryl-2-dipolyhydroxystearate;
oxyalkenylated organomodified silicone / polysiloxane / polyalkyl / polyether copolymers and derivatives.

Preferred emollients include
esters of linear or branched fatty acids with linear or branched fatty alcohols such as propylheptyl caprylate, coco caprylate, isopropyl myristate, ethylhexyl palmitate;
esters of aromatic carboxylic acids with linear or branched fatty alcohols such as C₁₂-C₁₅-alkyl benzoate, ethylhexyl benzoate, phenethyl benzoate;
dicarboxylic acid esters with linear or branched alcohols such as dibutyl adipate, dicaprylyl carbonate, diisopropyl sebacate;
esters of hydroxycarboxylic acids with linear or branched fatty alcohols;
esters of linear or branched fatty acids with polyhydric alcohol such as butylene glycol dicaprylate/dicaprate;
mono-, di-, tri-glycerides based on C₆-C₁₈ fatty acids such as caprylic / capric triglycerides, coco glycerides;
guerbet alcohols such as octyldodecynol;
hydrocarbons such as hydrogenated polyisobutene, mineral oil, squalene, isohexadecane;
ethers such as dicaprylyl ether;
silicone derivatives (organomodified polysiloxanes) such as dimethylpolysiloxane, cyclic silicones.

Preferred thickeners (also referred to as viscosity regulators) include
fatty alcohols such as cetyl alcohol, cetearyl alcohol, stearyl alcohol;
fatty acids such as stearic acid;
fatty acid esters such as myristyl stearate;
waxes such as beeswax, carnauba wax, microcrystalline wax, ceresin, ozocerite;
polysaccharides or derivatives such as xanthan gum, guar gum, agar gum, alginates, gellan gum, carraghenan;
polyacrylates or homopolymers of reticulated acrylic acids or polyacrylamides such as carbomers, acrylate copolymers, acrylate/C₁₀-C₃₀-alkyl acrylate crosspolymer, acrylate/beheneth-25 methacrylate copolymer;
silicate derivatives such as magnesium silicates;
cellulose derivatives such as hydroxypropyl cellulose.

In one embodiment of the present invention, the anti-aging care composition further comprises an additive selected from the group consisting of sensory enhancers, adjuvants, preservatives, and combinations thereof.

Preferred sensory enhancers include
polyamide derivatives such as nylon-12;
polymethyl methacrylates;
silica;
mica;
polymethylsilsesquioxane;
polyethylene;
starch derivatives such as aluminum starch octenylsuccinate;
dimethicone derivatives;
boron nitride;
HDI/trimethylol hexyllactone crosspolymer.

Preferred adjuvants include
tocopherol derivatives;
retinol derivatives;
ascorbic acid derivatives;
bisabolol;
allantoin;
panthenol;
chelating agents (EDTA, EDDS, EGTA, phytic acid, piroctone olamine);
ethylhexyl glycerin;
caprylyl glycol;
hydroxyacetophenone;
caprylhydroxymic acid;
propellants such as propane, butane, isobutene, dimethyl ether;
styrene/PVP or styrene acrylamide copolymers;
insect repellants such as butylacetylaminopropionate.

Preferred preservatives include
benzyl alcohol;
zingerone.

In connection with the above embodiments, it is to be understood that if the body-care composition (e.g. the anti-aging care composition) comprises two or more additives, combinations of the additives as defined above are also part of the invention.

In connection with the above embodiments, it is to be understood that the body-care composition (e.g. the anti-aging care composition), in its final formulation, may exist in a wide variety of presentation forms, which include
liquid preparations as a W/O, O/W, O/W/O, W/O/W or PIT emulsion and all kinds of micro emulsions;
gels;
oil, cream, milk or lotion;
powder, a lacquer, a tablet or make-up;
sticks;
sprays (spray with propellant gas or pump-action spray) or an aerosol;
foams;
pastes.

In one embodiment of the present invention, the anti-aging care composition is free of polyethylene glycol and polypropylene glycol.

In connection with the above preferred embodiments regarding the anti-aging care composition, in particular with regard to the UV filter DHHB and BMDBM, it has surprisingly been found by the inventors of the present invention that BM DBM remains stable in the composition. A skilled person is aware, that BM DBM is a frequently used UV absorber, which tends to isomerize under radiation to build a diketone in the triplet state. This diketone is very likely to undergo photolysis leading to a degradation of BMDBM. Therefore, stabilizing agents need to be added to compositions comprising BMDBM, for example photo stable UV absorber such as ethylhexyl-2-cyano-3,3-diphenyl-acrylate (INCI octocrylene) are commonly used. Thus, it has been a surprising finding by the inventors of the present invention, that BM DBM can be stabilized in an octocrylene free sunscreen composition by using the UV filter DHHB even in the presence of at least one perfume.

In one embodiment of the present invention, the anti-aging care composition is free of ethylhexyl methoxycinnamate and octocrylene.

In one embodiment of the present invention, the diethylamino hydroxybenzoyl hexyl benzoate in said anti-aging care composition comprising at least one perfume is used for reducing the lipid peroxidation, which is triggered by UV radiation.

In one embodiment of the present invention, the diethylamino hydroxybenzoyl hexyl benzoate in said anti-aging care composition comprising at least one perfume is used for reducing the aldehyde formation, which is triggered by UV radiation, preferably for reducing the formation of malondialdehyde.

In another embodiment the invention relates to a body-care composition comprising diethylamino hydroxybenzoyl hexyl benzoate and at least one perfume for use in a method of preventing skin reaction, preferably dermatosis and/or allergic reactions. Preferably, the body-care composition comprising diethylamino hydroxybenzoyl hexyl benzoate and at least one perfume is for use in a method of preventing dermatosis and/or allergic reactions caused by perfumes.

In one embodiment of the present invention, the body-care composition is a sunscreen.

In another embodiment of the present invention, the body-care composition is an anti-aging care composition.

In one embodiment of the present invention, the body-care composition further comprises butyl methoxydibenzoylmethane.

In one embodiment of the present invention, the body-care composition further comprises at least one additional UV filter. In this connection, suitable additional UV filter are mentioned above.

In one embodiment of the present invention, the at least one perfume is selected from the group consisting of limonene, citral, linalool, alpha isomethyl ionone, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzyl alcohol, benzyl benzoate, benzylcinnamate, benzyl salicylate, bergamot oil, orange oil, bitter orange oil, butylphenylmethylpropioal, cardamom oil, cedrol, cinnamaldehyde, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexyl cinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin, and mixtures thereof.

In one embodiment of the present invention, the at least one perfume is selected from the group consisting of geraniol, linalool, hydroxycitronellal, alpha isomethyl ionone, citral, coumarin, limonene, citronellol, benzyl benzoate, benzyl salicylate, hexyl cinnamaldehyde, vanillin, eugenol, orange oil, benzyl alcohol, bergamot oil, cinnamaldehyde, and mixtures thereof, preferably is selected from the group consisting of geraniol, linalool, alpha isomethyl ionone, citral, coumarin, citronellol, benzyl salicylate, hexyl cinnamaldehyde, vanillin, orange oil, benzyl alcohol, bergamot oil, cinnamaldehyde, and mixtures thereof, more preferably is selected from the group consisting of alpha isomethyl ionone, citral, coumarin, citronellol, benzyl salicylate, hexyl cinnamaldehyde, vanillin, orange oil, cinnamaldehyde, and mixtures thereof, and even more preferably is selected from the group consisting of alpha isomethyl ionone, citral, hexyl cinnamaldehyde, and mixtures thereof, and in particular is hexyl cinnamaldehyde.

In a preferred embodiment of the present invention, the least one perfume is selected from the group consisting of alpha isomethyl ionone, citral, coumarin, benzyl salicylate, hexyl cinnamaldehyde, vanillin, cinnamaldehyde, bitter orange oil, and mixtures thereof.

In a preferred embodiment of the present invention, the at least one perfume is selected from the group consisting of alpha isomethyl ionone, citral, coumarin, benzyl salicylate, hexyl cinnamaldehyde, vanillin, cinnamaldehyde, and mixtures thereof. In a preferred embodiment of the present invention, the at least one perfume is selected from the group consisting of alpha isomethyl ionone, citral, benzyl salicylate, hexyl cinnamaldehyde, vanillin, and mixtures thereof.

In a preferred embodiment of the present invention, the at least one perfume is selected from the group consisting of alpha isomethyl ionone, hexyl cinnamaldehyde, vanillin, and mixtures thereof. In a preferred embodiment of the present invention, the at least one perfume is selected from the group consisting of hexyl cinnamaldehyde, vanillin, and mixtures thereof.

In a preferred embodiment of the present invention, the at least one perfume is selected from the group consisting of geraniol, alpha isomethyl ionone, citral, coumarin, benzyl salicylate, hexyl cinnamaldehyde, eugenol, and mixtures thereof .

In a preferred embodiment of the present invention, the at least one perfume is selected from the group consisting of geraniol, alpha isomethyl ionone, citral, coumarin, benzyl salicylate, hexyl cinnamaldehyde, eugenol, and mixtures thereof.

In a preferred embodiment of the present invention, the perfume is alpha isomethyl ionone. In another preferred embodiment of the present invention, the at least one perfume is citral. In another preferred embodiment of the present invention, the at least one perfume is hexyl cinnamaldehyde. In another preferred embodiment of the present invention, the at least one perfume is geraniol. In another preferred embodiment of the present invention, the at least one perfume is coumarin. In another preferred embodiment of the present invention, the at least one perfume is benzyl salicylate. In another preferred embodiment of the present invention, the at least one perfume is eugenol.

In one embodiment of the present invention, the body-care composition comprises the at least one perfume in an amount of from 0.00001 to 1 wt.-%, preferably from 0.001 to 0.5 wt.-%, based on the total weight of the anti-aging care composition; diethylamino hydroxybenzoyl hexyl benzoate in an amount of from 0.1 to 10 wt.-%, preferably from 0.1 to 8 wt.-%, based on the total weight of the anti-aging care composition; optionally butyl methoxydibenzoylmethane in an amount of from 0.1 to 5 wt.-%, preferably from 0.1 to 4 wt.-%, based on the total weight of the anti-aging care composition; and optionally at least one additional UV filter in an amount of from 0.1 to 10 wt.-%, preferably from 0.1 to 8 wt.-%, based on the total weight of the anti-aging care composition.

In one embodiment of the present invention, the body-care composition comprises the at least one perfume in an amount of from 0.001 to 0.5 wt.-%, based on the total weight of the anti-aging care composition;

diethylamino hydroxybenzoyl hexyl benzoate in an amount of from 0.1 to 8 wt.-%, based on the total weight of the anti-aging care composition;

butyl methoxydibenzoylmethane in an amount of from 0.1 to 4 wt.-%, based on the total weight of the anti-aging care composition;

optionally at least one additional UV filter in an amount of from 0.1 to 8 wt.-%, based on the total weight of the anti-aging care composition.

In one embodiment of the present invention, the body-care composition further comprises a dermatologically acceptable emulsifier, thickener, and/or emollient.

In one embodiment of the present invention, the body-care composition is free of polyethylene glycol and polypropylene glycol.

In one embodiment of the present invention, the body-care composition is free of ethylhexyl methoxycinnamate and octocrylene.

In one embodiment of the present invention, the skin reaction such as dermatosis and/or allergic reactions is prevented by reduction of oxidative degradation, preferably by reduction of lipid peroxidation. In one embodiment of the present invention, the skin reaction such as dermatosis and/or allergic reactions is prevented by reduction of lipid peroxidation, which is triggered by UV radiation. In one embodiment of the present invention, the skin reaction such as dermatosis and/or allergic reactions is prevented by reduction of the aldehyde formation, which is triggered by UV radiation, preferably the formation of malondialdehyde is reduced.

In a preferred embodiment of the present invention dermatosis and/or allergic reactions is prevented by reduction of oxidative degradation, preferably by reduction of lipid peroxidation, wherein the dermatosis and/or allergic reactions is caused by perfumes. In a preferred embodiment of the present invention dermatosis and/or allergic reactions is prevented by reduction of lipid peroxidation, which is triggered by UV radiation, wherein the dermatosis and/or allergic reactions is caused by perfumes. In a preferred embodiment of the present invention, dermatosis and/or allergic reactions is prevented by reduction of the aldehyde formation, which is triggered by UV radiation, preferably the formation of malondialdehyde is reduced, wherein the dermatosis and/or allergic reactions is caused by perfumes.

In another embodiment, the present invention relates to a photostable body-care composition comprising diethylamino hydroxybenzoyl hexyl benzoate and at least one perfume. It is to be understood that the photostable body-care composition stays stable upon UV irradiation, in particular that the therein comprised compounds do not undergo a degradation process upon UV radiation.

The present invention is further illustrated by the following examples.

### EXAMPLES

Methods

The Electron Spin Resonance (ESR) measurements were performed with a high sensitive X-band bench top Electron Spin Resonance Spectrometer MiniScope MS300, supplied from Magnettech GmbH Berlin, Germany.

The UV irradiation of the samples was performed with a xenon arc lamp Solar Simulator from Newport-ORIEL Product Line 81260 (US,Newport Solar Simulators—productspecifications) equipped with a 300 W Xenon lamp.

2,2,5,5,-tetramethyl pyrrolidine N-oxyl was purchased from Merck.

Synthetic sebum was purchased from wfk Testgewebe GmbH (Bruggen, DE).

### Example 1

### Determination of the Free Radicals Generated Inside a Formulation Containing Perfumes

To measure the free radicals generation inside the formulation after UV irradiation, the concentration of a photostable spin marker added to the tested samples was followed by

Electron Spin Resonance (ESR) spectroscopy before and after UV irradiation of the tested samples. The photostable spin marker promptly reacted with the free radical generated in the formulation after UV irradiation.

0.1% (w/w) of the perfume to be tested (compare to Table 1) was incorporated into the formulation. The formulation was then diluted in distillated water (1:10 dilution factor) and a water solution of the spin marker PCA (2,2,5,5,-tetramethyl pyrrolidine N-oxyl) was added to obtain a spin marker concentration of 0.01 mM. Knowing the concentration of the spin trap PCA within the formulation, the amount of reduced PCA was calculated. The sample was inserted in a capillary quartz tube to perform ESR measurement before and after 20 minutes of UV irradiation. The irradiances as integrated value over the spectral ranges of the solar simulator were E=23,5 W/m² for UVB and 180 W/m² for UVA.

The percentage of UV-induced free radicals inside the formulation containing said tested perfume is given in Table 1.

TABLE 1
percentage of UV-induced free radicals in
the presence of the respective perfume

| | | % free radicals |
|---|---|---|
| | | after 20 min UV |
| Perfume | CAS | irradiation |
| Geraniol | 106-24-1 | 4.9 |
| Linalool | 78-70-6 | 4.6 |
| Hydroxycitronellal | 107-75-5 | 3.7 |
| Alpha isomethyl ionone (alpha cetone) | 127-51-5 | 22.4 |
| Citral | 5392-40-5 | 16.5 |
| Coumarin | 91-64-5 | 11.5 |
| Limonene | 5989-27-5 | 1.9 |
| Citronellol | 106-22-9 | 6.6 |
| Benzyl Benzoate | 120-51-4 | 3.4 |
| Benzyl Salicylate | 118-58-1 | 17.5 |
| Hexyl cinnamaldehyde | 101-86-0 | 75.7 |
| Vanillin | 121-33-5 | 85.9 |
| Eugenol | 97-53-0 | 2.6 |
| Orange oil | 8028-48-6 | 8.4 |
| Benzyl Alcohol | 100-51-6 | 4.4 |
| Bergamot oil | 8007-75-8 | 4.8 |
| cinnamaldehyde | 104-55-2 | 10.5 |

### Example 2

### Impact of the Uv Filter System to Reduce Sebum Oxidation Induced by Perfumes

Squalene in sebum is highly sensitive to oxidation. Squalene by-products in peroxidized form impact cellular and tissue physiologies. In this lipid oxidative degradation, free radicals take electrons from the lipid. Lipid peroxidation forms reactive aldehydes such as malondialdehyde (MDA) as natural by-product and can be used as marker of lipid peroxidation to assess oxidative damage.

Based on this principle the following in vitro method was developed to evaluate the sebum lipid oxidation.

The tested formulations were manufactured as follows.

The ingredients of part A, as well as the ingredients of part B as provided below in Tables 2.1, 2.2, 2.3 and 2.4 for each tested sunscreen composition were combined and heated to 80 ° C. respectively, wherein part A was added to part B under stirring and was further homogenized. Subsequently, the composition was cooled down to room temperature under stirring. If present, parts C, D, and E were added under stirring and adjust the pH of each composition to 5.5-7.00 with NaOH (30%) if necessary.

The remaining perfumes from Table 1 were incorporated into the formulation according to Table 2.1.

TABLE 2.1
formulations without UV filter

| | Comp. | Comp. | Comp. | Comp. |
|---|---|---|---|---|
| Ingredient (Trade Name) | 1 | 2 | 3 | 4 |

Part A

| | | | | |
|---|---|---|---|---|
| Sucrose Polystearate (and) | 3.00 | 3.00 | 3.00 | 3.00 |
| Hydrogenated Polyisobutene | | | | |
| (Emulgade Sucro) | | | | |
| Disodium Cetearyl Sulfosuccinate | 1.00 | 1.00 | 1.00 | 1.00 |
| (Eumulgin Prisma) | | | | |
| Cetearyl Alcohol (Lanette O) | 1.00 | 1.00 | 1.00 | 1.00 |
| Dicaprylyl Ether (Cetiol OE) | 3.00 | 3.00 | 3.00 | 3.00 |
| Propylheptyl Caprylate (Cetiol | 5.00 | 5.00 | 5.00 | 5.00 |
| Sensoft) | | | | |
| Dibutyl Adipate (Cetiol B) | 10.00 | 10.00 | 10.00 | 10.00 |
| Phenoxyethanol and | 1.00 | 1.00 | 1.00 | 1.00 |
| Ethylhexylglycerin | | | | |

Part B

| | | | | |
|---|---|---|---|---|
| Water | qsp | qsp | qsp | qsp |
| | 100% | 100% | 100% | 100% |
| Glycerine | 2.00 | 2.00 | 2.00 | 2.00 |
| Xanthan Gum | 0.20 | 0.20 | 0.20 | 0.20 |
| Disodium EDTA | 0.20 | 0.20 | 0.20 | 0.20 |

Part C

| | | | | |
|---|---|---|---|---|
| Undecane, Tridecane (Cetiol | 2.00 | 2.00 | 2.00 | 2.00 |
| Ultimate) | | | | |

Part D

| | |
|---|---|
| Ethanol Abs. | 2.00 |

Part E

| | | | |
|---|---|---|---|
| Hexylcinnamaldehyde Sol. 0.5% in | 2.00 | | |
| EtOH Abs. (0.01% ai) | | | |
| Alpha cetone Sol 0.5% in EtOH | | 2.00 | |
| Abs. (0.01% ai) | | | |
| Citral Sol 0.5% in EtOH Abs. | | | 2.00 |
| (0.01% ai) | | | |

TABLE 2.2
formulations with BMDBM

| | | | |
|---|---|---|---|
| Ingredient (Trade Name) | Comp. 5 | Comp. 6 | Comp. 7 |

Part A

| | | | |
|---|---|---|---|
| Sucrose Polystearate (and) | 3.00 | 3.00 | 3.00 |
| Hydrogenated Polyisobutene | | | |
| (Emulgade Sucro) | | | |
| Disodium Cetearyl Sulfosuccinate | 1.00 | 1.00 | 1.00 |
| (Eumulgin Prisma) | | | |
| Cetearyl Alcohol (Lanette O) | 1.00 | 1.00 | 1.00 |
| Dicaprylyl Ether (Cetiol OE) | 3.00 | 3.00 | 3.00 |
| Propylheptyl Caprylate (Cetiol | 5.00 | 5.00 | 5.00 |
| Sensoft) | | | |
| Dibutyl Adipate (Cetiol B) | 10.00 | 10.00 | 10.00 |
| Phenoxyethanol and | 1.00 | 1.00 | 1.00 |
| Ethylhexylglycerin | | | |
| Butyl Methoxydibenzolmethane | 2.00 | 2.00 | 2.00 |

Part B

| | | | |
|---|---|---|---|
| Water | qsp 100% | qsp 100% | qsp 100% |
| Glycerine | 2.00 | 2.00 | 2.00 |
| Xanthan Gum | 0.20 | 0.20 | 0.20 |
| Disodium EDTA | 0.20 | 0.20 | 0.20 |

Part C

| | | | |
|---|---|---|---|
| Undecane, Tridecane (Cetiol | 2.00 | 2.00 | 2.00 |
| Ultimate) | | | |

Part E

| | | | |
|---|---|---|---|
| Hexylcinnamaldehyde Sol. 0.5% in | 2.00 | | |
| EtOH Abs. (0.01% ai) | | | |
| Alpha cetone Sol 0.5% in EtOH | | 2.00 | |
| Abs. (0.01% ai) | | | |
| Citral Sol 0.5% in EtOH Abs. | | | 2.00 |
| (0.01% ai) | | | |

TABLE 2.3
formulations without UV filter

| | Comp. | Comp. | Comp. | Comp. |
|---|---|---|---|---|
| Ingredient (Trade Name) | 8 | 9 | 10 | 11 |

Part A

| | | | | |
|---|---|---|---|---|
| Sucrose Polystearate (and) | 3.00 | 3.00 | 3.00 | 3.00 |
| Hydrogenated Polyisobutene | | | | |
| (Emulgade Sucro) | | | | |
| Disodium Cetearyl Sulfosuccinate | 1.00 | 1.00 | 1.00 | 1.00 |
| (Eumulgin Prisma) | | | | |
| Cetearyl Alcohol (Lanette O) | 1.00 | 1.00 | 1.00 | 1.00 |
| Dicaprylyl Ether (Cetiol OE) | 3.00 | 3.00 | 3.00 | 3.00 |
| Propylheptyl Caprylate (Cetiol | 5.00 | 5.00 | 5.00 | 5.00 |
| Sensoft) | | | | |
| Dibutyl Adipate (Cetiol B) | 10.00 | 10.00 | 10.00 | 10.00 |
| Phenoxyethanol and | 1.00 | 1.00 | 1.00 | 1.00 |
| Ethylhexylglycerin | | | | |

Part B

| | | | | |
|---|---|---|---|---|
| Water | qsp | qsp | qsp | qsp |
| | 100% | 100% | 100% | 100% |
| Glycerine | 2.00 | 2.00 | 2.00 | 2.00 |
| Xanthan Gum | 0.20 | 0.20 | 0.20 | 0.20 |
| Disodium EDTA | 0.20 | 0.20 | 0.20 | 0.20 |

Part C

| | | | | |
|---|---|---|---|---|
| Undecane, Tridecane (Cetiol | 2.00 | 2.00 | 2.00 | 2.00 |
| Ultimate) | | | | |

Part E

| | | | | |
|---|---|---|---|---|
| Coumarin Sol. 0.5% in EtOH Abs. | 2.00 | | | |
| (0.01% ai) | | | | |
| Eugenol Sol 0.5% in EtOH Abs. | | 2.00 | | |
| (0.01% ai) | | | | |
| Benzyl salicylate Sol 0.5% in | | | 2.00 | |
| EtOH Abs. (0.01% ai) | | | | |
| Geraniol Sol 0.5% in EtOH Abs. | | | | 2.00 |
| (0.01% ai) | | | | |

TABLE 2.4
formulations with DHHB

| | Inv. | Inv. | Inv. | Inv. |
|---|---|---|---|---|
| Ingredient (Trade Name) | 1 | 2 | 3 | 4 |

Part A

| | | | | |
|---|---|---|---|---|
| Sucrose Polystearate (and) | 3.00 | 3.00 | 3.00 | 3.00 |
| Hydrogenated Polyisobutene | | | | |
| (Emulgade Sucro) | | | | |
| Disodium Cetearyl Sulfosuccinate | 1.00 | 1.00 | 1.00 | 1.00 |
| (Eumulgin Prisma) | | | | |
| Cetearyl Alcohol (Lanette O) | 1.00 | 1.00 | 1.00 | 1.00 |
| Dicaprylyl Ether (Cetiol OE) | 3.00 | 3.00 | 3.00 | 3.00 |
| Propylheptyl Caprylate (Cetiol | 5.00 | 5.00 | 5.00 | 5.00 |
| Sensoft) | | | | |
| Dibutyl Adipate (Cetiol B) | 10.00 | 10.00 | 10.00 | 10.00 |
| Phenoxyethanol and | 1.00 | 1.00 | 1.00 | 1.00 |
| Ethylhexylglycerin | | | | |
| Butyl Methoxydibenzolmethane | | | | 2.00 |
| Diethylamino Hydroxybenzoyl Hexyl | 2.00 | 2.00 | 2.00 | 2.00 |
| Benzoate (Uvinul A Plus) | | | | |

Part B

| | | | | |
|---|---|---|---|---|
| Water | qsp | qsp | qsp | qsp |
| | 100% | 100% | 100% | 100% |
| Glycerine | 2.00 | 2.00 | 2.00 | 2.00 |
| Xanthan Gum | 0.20 | 0.20 | 0.20 | 0.20 |
| Disodium EDTA | 0.20 | 0.20 | 0.20 | 0.20 |

Part C

| | | | | |
|---|---|---|---|---|
| Undecane, Tridecane (Cetiol | 2.00 | 2.00 | 2.00 | 2.00 |
| Ultimate) | | | | |

Part E

| | | | | |
|---|---|---|---|---|
| Hexylcinnamaldehyde Sol. 0.5% in | 2.00 | | | 2.00 |
| EtOH Abs. (0.01% ai) | | | | |
| Alpha cetone Sol 0.5% in EtOH | | 2.00 | | |
| Abs. (0.01% ai) | | | | |
| Citral Sol 0.5% in EtOH Abs. | | | 2.00 | |
| (0.01% ai) | | | | |

TABLE 2.5
formulations with DHHB

| | Inv. | Inv. | Inv. | Inv. |
|---|---|---|---|---|
| Ingredient (Trade Name) | 5 | 6 | 7 | 8 |

Part A

| | | | | |
|---|---|---|---|---|
| Sucrose Polystearate (and) | 3.00 | 3.00 | 3.00 | 3.00 |
| Hydrogenated Polyisobutene | | | | |
| (Emulgade Sucro) | | | | |
| Disodium Cetearyl Sulfosuccinate | 1.00 | 1.00 | 1.00 | 1.00 |
| (Eumulgin Prisma) | | | | |
| Cetearyl Alcohol (Lanette O) | 1.00 | 1.00 | 1.00 | 1.00 |
| Dicaprylyl Ether (Cetiol OE) | 3.00 | 3.00 | 3.00 | 3.00 |
| Propylheptyl Caprylate (Cetiol | 5.00 | 5.00 | 5.00 | 5.00 |
| Sensoft) | | | | |
| Dibutyl Adipate (Cetiol B) | 10.00 | 10.00 | 10.00 | 10.00 |
| Phenoxyethanol and | 1.00 | 1.00 | 1.00 | 1.00 |
| Ethylhexylglycerin | | | | |
| Butyl Methoxydibenzolmethane | 2.00 | 1.00 | 1.00 | 1.00 |
| Diethylamino Hydroxybenzoyl Hexyl | 2.00 | 1.00 | 1.00 | 1.00 |
| Benzoate (Uvinul A Plus) | | | | |

Part B

| | | | | |
|---|---|---|---|---|
| Water | qsp | qsp | qsp | qsp |
| | 100% | 100% | 100% | 100% |
| Glycerine | 2.00 | 2.00 | 2.00 | 2.00 |
| Xanthan Gum | 0.20 | 0.20 | 0.20 | 0.20 |
| Disodium EDTA | 0.20 | 0.20 | 0.20 | 0.20 |

Part C

| | | | | |
|---|---|---|---|---|
| Undecane, Tridecane (Cetiol | 2.00 | 2.00 | 2.00 | 2.00 |
| Ultimate) | | | | |

Part E

| | | | | |
|---|---|---|---|---|
| Hexylcinnamaldehyde Sol.0.5% in | | 2.00 | | |
| EtOH Abs. (0.01% ai) | | | | |
| Alpha cetone Sol 0.5% in EtOH | 2.00 | | 2.00 | |
| Abs. (0.01% ai) | | | | |
| Citral Sol 0.5% in EtOH Abs. | | | | 2.00 |
| (0.01% ai) | | | | |

TABLE 2.6
formulations with DHHB

| | Inv. | Inv. | Inv. | Inv. | Inv. |
|---|---|---|---|---|---|
| Ingredient (Trade Name) | 9 | 10 | 11 | 12 | 13 |

Part A

| | | | | | |
|---|---|---|---|---|---|
| Sucrose Polystearate (and) | 3.00 | 3.00 | 3.00 | 3.00 | 3.00 |
| Hydrogenated Polyisobutene | | | | | |
| (Emulgade Sucro) | | | | | |
| Disodium Cetearyl Sulfosuccinate | 1.00 | 1.00 | 1.00 | 1.00 | 1.00 |
| (Eumulgin Prisma) | | | | | |
| Cetearyl Alcohol (Lanette O) | 1.00 | 1.00 | 1.00 | 1.00 | 1.00 |
| Dicaprylyl Ether (Cetiol OE) | 3.00 | 3.00 | 3.00 | 3.00 | 3.00 |
| Propylheptyl Caprylate (Cetiol | 5.00 | 5.00 | 5.00 | 5.00 | 5.00 |
| Sensoft) | | | | | |
| Dibutyl Adipate (Cetiol B) | 10.00 | 10.00 | 10.00 | 10.00 | 10.00 |
| Phenoxyethanol and | 1.00 | 1.00 | 1.00 | 1.00 | 1.00 |
| Ethylhexylglycerin | | | | | |
| Butyl Methoxydibenzolmethane | | | | | 2.00 |
| Diethylamino Hydroxybenzoyl Hexyl | 2.00 | 2.00 | 2.00 | 2.00 | 2.00 |
| Benzoate (Uvinul A Plus) | | | | | |

Part B

| | | | | | |
|---|---|---|---|---|---|
| Water | qsp | qsp | qsp | qsp | qsp |
| | 100% | 100% | 100% | 100% | 100% |
| Glycerine | 2.00 | 2.00 | 2.00 | 2.00 | 2.00 |
| Xanthan Gum | 0.20 | 0.20 | 0.20 | 0.20 | 0.20 |
| Disodium EDTA | 0.20 | 0.20 | 0.20 | 0.20 | 0.20 |

Part C

| | | | | | |
|---|---|---|---|---|---|
| Undecane, Tridecane (Cetiol | 2.00 | 2.00 | 2.00 | 2.00 | 2.00 |
| Ultimate) | | | | | |

Part E

| | | | | | |
|---|---|---|---|---|---|
| Coumarin Sol. 0.5% in EtOH Abs. | 2.00 | | | | |
| (0.01% ai) | | | | | |
| Eugenol Sol 0.5% in EtOH Abs. | | 2.00 | | | |
| (0.01% ai) | | | | | |
| Benzyl salicylate Sol 0.5% in | | | 2.00 | | |
| EtOH Abs. (0.01% ai) | | | | | |
| Geraniol Sol 0.5% in EtOH Abs. | | | | 2.00 | |
| (0.01% ai) | | | | | |
| Citral Sol 0.5% in EtOH Abs. | | | | | 2.00 |
| (0.01% ai) | | | | | |

The tested formulation was applied in an amount of 2 mg/cm² on an artificial sebum film in a petri dish. The sebum film was formerly prepared by adding 1 g of synthetic sebum to 100 mL of heptane to obtain a sebum solution. 1 mL of this sebum solution was deposited onto a petri dish (diameter 6 cm) and put into a nitrogen chamber until complete solvent evaporation. The formulation film was allowed to dry in the nitrogen chamber and was then irradiated for 2 h, UVA at 1.25mW/cm².

The sebum was collected by adding heptane into the petri dish and using ultrasonic bath to dissolve the sebum film. Finally, the MDA level corresponding to the oxidation product from the sebum was determined using TBARS (thiobarbituric acid reactive substances) method. The MDA in the sample reacted with thiobarbituric acid (TBA) to generate a MDA-TBA adduct. The MDA-TBA adduct was quantified colorimetrically at 532 nm.

In this connection, 0.3 mL of the sebum samples solution, 0.2 mL 8 w% SDS solution, 1.5 mL 20 v% acetic acid, 1.5 mL TBA solution, and 0.5 mL D.I. water was added into a tub, the mixture was then heated at 95 ° C. for 60 minutes, cooled down to room temperature in an ice bath for 15 minutes, and centrifuged at 3000 rpm for 5 minutes. The absorbance of the reaction solution was measured at 532 nm by an UV spectrophotomer to detect the presence of TBA-MDA adduct.

TABLE 3
detected MDA-level of the tested formulations; #—test
series 1; *test series 2; n.d.—not determined

| | | MDA | |
|---|---|---|---|
| | | level | |
| | | (μg/mg | |
| No. | condition | sebum) | observation |
| Comp. 1 | #No UV filters, no | 2.14 | Oxidation level of the |
| | perfume | | placebo |
| Comp. 2 | #No UV filters, 0.01% | 2.81 | Perfume increases |
| | Hexyl cinnamaldehyde | | sebum oxidation level |
| Comp. 3 | #No UV filters, 0.01% | 2.68 | Perfume increases |
| | Alpha isomethyl ionone | | sebum oxidation level |
| | (alpha cetone) | | |
| Comp. 4 | #No UV filters, 0.01% | 2.56 | Perfume increases |
| | citral | | sebum oxidation level |
| Comp. 5 | #2% BMDBM, 0.01% | 1.99 | UVA filter BMDBM |
| | Hexyl cinnamaldehyde | | reduces oxidation from |
| | | | perfume |
| Comp. 6 | #2% BMDBM, 0.01% | 1.43 | UVA filter BMDBM |
| | Alpha isomethyl ionone | | reduces oxidation from |
| | (alpha cetone) | | perfume |
| Comp. 7 | #2% BMDBM, 0.01% | 1.37 | UVA filter BMDBM |
| | citral | | reduces oxidation from |
| | | | perfume |
| Comp. 8 | *No UV filters, 0.01% | 1.59 | |
| | coumarin | | |
| Comp. 9 | *No UV filters, 0.01% | 2.09 | |
| | eugenol | | |
| Comp. 10 | *No UV filters, 0.01% | 1.46 | |
| | benzyl salicylate | | |
| Comp. 11 | *No UV filters, 0.01% | 1.37 | |
| | geraniol | | |
| Inv. 1 | #2% DHHB, 0.01% | 1.42 | UVA filter DHHB |
| | Hexyl cinnamaldehyde | | increases reduction of |
| | | | oxidation from perfume |
| Inv. 2 | #2% DHHB, 0.01% | 0.97 | UVA filter DHHB |
| | Alpha isomethyl | | increases reduction of |
| | ionone (alpha cetone) | | oxidation from perfume |
| Inv. 3 | #2% DHHB, 0.01% | 1.13 | UVA filter DHHB |
| | citral | | increases reduction of |
| | | | oxidation from perfume |
| Inv. 4 | #2% BMDBM, 2% | 0.77 | UVA filter combination |
| | DHHB, 0.01% Hexyl | | DHHB and BMDBM |
| | cinnamaldehyde | | increases reduction of |
| | | | oxidation from perfume |
| Inv. 5 | #2% BMDBM, 2% | 1.08 | UVA filter combination |
| | DHHB, 0.01% Alpha | | DHHB and BMDBM |
| | isomethyl ionone | | increases reduction of |
| | (alpha cetone) | | oxidation from perfume |
| Inv. 6 | #1% BMDBM, 1% | 1.15 | UVA filter combination |
| | DHHB, 0.01% Hexyl | | DHHB and BMDBM |
| | cinnamaldehyde | | increases reduction of |
| | | | oxidation from perfume |
| Inv. 7 | #1% BMDBM, 1% | 1.12 | UVA filter combination |
| | DHHB, 0.01% Alpha | | DHHB and BMDBM |
| | isomethyl ionone | | increases reduction of |
| | (alpha cetone) | | oxidation from perfume |
| Inv. 8 | #1% BMDBM, 1% | 1.23 | UVA filter combination |
| | DHHB, 0.01% | | DHHB and BMDBM |
| | citral | | increases reduction of |
| | | | oxidation from perfume |
| Inv. 9 | *2% DHHB, 0.01% | 1.04 | UVA filter DHHB |
| | coumarin | | decreases sebum |
| | | | oxidation level further |
| Inv. 10 | *2% DHHB, 0.01% | 1.02 | UVA filter DHHB |
| | eugenol | | decreases sebum |
| | | | oxidation level further |
| Inv. 11 | *2% DHHB, 0.01% | 0.60 | UVA filter DHHB |
| | benzyl salicylate | | decreases sebum |
| | | | oxidation level further |
| Inv. 12 | *2% DHHB, 0.01% | 0.68 | UVA filter DHHB |
| | geraniol | | decreases sebum |
| | | | oxidation level further |
| Inv. 13 | *2% BMDBM, 2% | n.d. | |
| | DHHB, 0.01% citral | | |

As can be seen from Table 3, the lowest MDA level is achieved, when the formulation comprises DHHB.

1.-15. (canceled)

16. An anti-aging care composition comprising diethylamino hydroxybenzoyl hexyl benzoate and at least one perfume.

17. The composition according to claim 16, for reducing the oxidative degradation.

18. The composition according to claim 16, for reducing the lipid peroxidation.

19. The composition according to claim 16, wherein the anti-aging care composition further comprises butyl methoxydibenzoylmethane and/or at least one additional UV filter.

20. The composition according to claim 16, wherein the at least one perfume is selected from the group consisting of limonene, citral, linalool, alpha isomethyl ionone, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amyl salicylate, amylcinnamylalcohol, anisalcohol, benzoin, benzyl alcohol, benzyl benzoate, benzylcinnamate, benzyl salicylate, bergamot oil, orange oil, bitter orange oil, butylphenylmethylpropioal, cardamom oil, cedrol, cinnamaldehyde, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexyl cinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin, and mixtures thereof

21. The composition according to claim 16, wherein the at least one perfume is selected from the group consisting of geraniol, linalool, alpha isomethyl ionone, citral, coumarin, citronellol, benzyl salicylate, hexyl cinnamaldehyde, vanillin, orange oil, benzyl alcohol, bergamot oil, cinnamaldehyde, and mixtures thereof.

22. The composition according to claim 16, wherein the anti-aging care composition comprises hexyl cinnamaldehyde, diethylamino hydroxybenzoyl hexyl benzoate, optionally butyl methoxydibenzoylmethane, and optionally at least one additional UV filter.

23. The composition according to claim 16, wherein the anti-aging care composition comprises the at least one perfume, diethylamino hydroxybenzoyl hexyl benzoate, butyl methoxydibenzoylmethane, and optionally at least one additional UV filter.

24. The composition according to claim 16, wherein the anti-aging care composition comprises
the at least one perfume in an amount of from 0.00001 to 1 wt.-%, based on the total weight of the anti-aging care composition;
diethylamino hydroxybenzoyl hexyl benzoate in an amount of from 0.1 to 10 wt.-%, based on the total weight of the anti-aging care composition;
optionally butyl methoxydibenzoylmethane in an amount of from 0.1 to 5 wt.-%, based on the total weight of the anti-aging care composition; and
optionally at least one additional UV filter in an amount of from 0.1 to 10 wt.-%, based on the total weight of the anti-aging care composition.

25. The composition according to claim 16, wherein the anti-aging care composition comprises
the at least one perfume in an amount of from 0.001 to 0.5 wt.-%, based on the total weight of the anti-aging care composition;
diethylamino hydroxybenzoyl hexyl benzoate in an amount of from 0.1 to 8 wt.-%, based on the total weight of the anti-aging care composition;
butyl methoxydibenzoylmethane in an amount of from 0.1 to 4 wt.-%, based on the total weight of the anti-aging care composition;
optionally at least one additional UV filter in an amount of from 0.1 to 8 wt.-%, based on the total weight of the anti-aging care composition.

26. The composition according to claim 16, wherein the anti-aging care composition further comprises a dermatologically acceptable emulsifier, thickener, and/or emollient.

27. The composition according to claim 16, wherein the anti-aging care composition is free of polyethylene glycol and polypropylene glycol and/or is free of ethylhexyl methoxycinnamate and octocrylene.

28. The composition according to claim 16, for reducing the lipid peroxidation, which is triggered by UV radiation.

29. The composition according to claim 16, for reducing the aldehyde formation, which is triggered by UV radiation, preferably for reducing the formation of malondialdehyde.

30. A body-care composition comprising diethylamino hydroxybenzoyl hexyl benzoate and at least one perfume for use in a method of preventing dermatosis and/or allergic reactions caused by perfumes.
